(19) United States
(12) Patent Application Publication
(10) Pub. No.: US 2018/0263677 A1
(43) Pub. Date: Sep. 20, 2018

(54) ADHESIVE LINERS FOR CRYOTHERAPY

(71) Applicant: Zeltiq Aesthetics, Inc., Pleasanton, CA (US)

(72) Inventors: Tamara Lynn Hilton, Alameda, CA (US); Joseph Coakley, Dublin, CA (US)

(21) Appl. No.: 15/921,869

(22) Filed: Mar. 15, 2018

Related U.S. Application Data
(60) Provisional application No. 62/472,416, filed on Mar. 16, 2017.

(51) Int. Cl. A61B 18/02; A61F 7/00; A61F 7/02

(52) U.S. Cl. CPC A61B 18/02; A61F 7/0085; A61F 7/02; A61F 2007/0001; A61F 2007/029; A61F 2007/0056; A61F 2007/0226

(57) ABSTRACT

Treatment systems include conformable applicators and adhesive liners for performing cryotherapy. Aspects of the technology are further directed to treatment methods and conformable applicators capable of affecting target regions. The conformable applicators and liners can be applied to a wide range of treatment sites. The liner can include a thermal coupling substance within a sealed reservoir to promote thermal coupling with the target region. A strap assembly can hold the applicator against the treatment site, and a holder assembly can be adhered to the subject's skin to inhibit, limit, or substantially prevent movement of the applicator along the subject's skin.

### CROSS-REFERENCE TO RELATED APPLICATION

The present application claims the benefit of and priority under 35 U.S.C. § 119(e) to U.S. Provisional Patent Application No. 62/472,616, filed Mar. 16, 2017, which is incorporated herein by reference in its entirety.

### INCORPORATION BY REFERENCE OF APPLICATIONS AND PATENTS

The following commonly assigned U.S. patent applications and U.S. patents are incorporated herein by reference in their entirety:

U.S. Patent Publication No. 2008/0287839 entitled “METHOD OF ENHANCED REMOVAL OF HEAT FROM SUBCUTANEOUS LIPID-RICH CELLS AND TREATMENT APPARATUS HAVING AN ACTUATOR”;

U.S. Pat. No. 6,032,675 entitled “FREEZING METHOD FOR CONTROLLED REMOVAL OF FATTY TISSUE BY LIPOSUCTION”;

U.S. Patent Publication No. 2007/0255362 entitled “CRYOPROTECTANT FOR USE WITH A TREATMENT DEVICE FOR IMPROVED COOLING OF SUBCUTANEOUS LIPID-RICH CELLS”;

U.S. Pat. No. 7,854,754 entitled “COOLING DEVICE FOR REMOVING HEAT FROM SUBCUTANEOUS LIPID-RICH CELLS”;

U.S. Pat. No. 8,337,539 entitled “COOLING DEVICE FOR REMOVING HEAT FROM SUBCUTANEOUS LIPID-RICH CELLS”;

U.S. Patent Publication No. 2008/0077201 entitled “COOLING DEVICES WITH FLEXIBLE SENSORS”;

U.S. Pat. No. 9,132,031 entitled “COOLING DEVICE HAVING A PLURALITY OF CONTROLLABLE COOLING ELEMENTS TO PROVIDE A PREDETERMINED COOLING PROFILE”;

U.S. Patent Publication No. 2009/0118722, filed Oct. 31, 2007, entitled “METHOD AND APPARATUS FOR COOLING SUBCUTANEOUS LIPID-RICH CELLS OR TISSUE”;

U.S. Patent Publication No. 2009/0018624 entitled “LIMITING USE OF DISPOSABLE SYSTEM PATIENT PROTECTION DEVICES”;

U.S. Pat. No. 8,523,927 entitled “SYSTEM FOR TREATING LIPID-RICH REGIONS”;

U.S. Patent Publication No. 2009/0018625 entitled “MANAGING SYSTEM TEMPERATURE TO REMOVE HEAT FROM LIPID-RICH REGIONS”;

U.S. Patent Publication No. 2009/0018627 entitled “SECURE SYSTEM FOR REMOVING HEAT FROM LIPID-RICH REGIONS”;

U.S. Patent Publication No. 2009/0018626 entitled “USER INTERFACES FOR A SYSTEM THAT REMOVES HEAT FROM LIPID-RICH REGIONS”;

U.S. Pat. No. 6,041,787 entitled “USE OF CRYOPROTECTIVE AGENT COMPOUNDS DURING CRYOSURGERY”;

U.S. Pat. No. 8,285,390 entitled “MONITORING THE COOLING OF SUBCUTANEOUS LIPID-RICH CELLS, SUCH AS THE COOLING OF ADIPOSE TISSUE”;

U.S. Provisional Patent Application Ser. No. 60/941,567 entitled “METHODS, APPARATUSES AND SYSTEMS FOR COOLING THE SKIN AND SUBCUTANEOUS TISSUE”;

U.S. Pat. No. 8,275,442 entitled “TREATMENT PLANNING SYSTEMS AND METHODS FOR BODY CONTOURING APPLICATIONS”;

U.S. patent application Ser. No. 12/275,002 entitled “APPARATUS WITH HYDROPHILIC RESERVOIRS FOR COOLING SUBCUTANEOUS LIPID-RICH CELLS”;

U.S. patent application Ser. No. 12/275,014 entitled “APPARATUS WITH HYDROPHOBIC FILTERS FOR REMOVING HEAT FROM SUBCUTANEOUS LIPID-RICH CELLS”;

U.S. Pat. No. 8,603,073 entitled “SYSTEMS AND METHODS WITH INTERRUPT/RESUME CAPABILITIES FOR COOLING SUBCUTANEOUS LIPID-RICH CELLS”;

U.S. Pat. No. 8,192,474 entitled “TISSUE TREATMENT METHODS”;

U.S. Pat. No. 8,702,774 entitled “DEVICE, SYSTEM AND METHOD FOR REMOVING HEAT FROM SUBCUTANEOUS LIPID-RICH CELLS”;

U.S. Pat. No. 8,676,388 entitled “COMBINED MODALITY TREATMENT SYSTEMS, METHODS AND APPARATUS FOR BODY CONTOURING APPLICATIONS”;

U.S. Pat. No. 9,314,368 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;

U.S. Pat. No. 9,844,461 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;

U.S. Publication No. 2012/0239123 entitled “DEVICES, APPLICATION SYSTEMS AND METHODS WITH LOCALIZED HEAT FLUX ZONES FOR REMOVING HEAT FROM SUBCUTANEOUS LIPID-RICH CELLS”:

U.S. Pat. No. 9,545,523 entitled “MULTI-MODALITY TREATMENT SYSTEMS, METHODS AND APPARATUS FOR ALTERING SUBCUTANEOUS LIPID-RICH TISSUE”;

U.S. Pat. No. 9,844,460 entitled “TREATMENT SYSTEMS WITH FLUID MIXING SYSTEMS AND FLUID-COOLED APPLICATORS AND METHODS OF USING THE SAME”;

U.S. Pat. No. 9,132,031 entitled “COOLING DEVICE HAVING A PLURALITY OF CONTROLLABLE COOLING ELEMENTS TO PROVIDE A PREDETERMINED COOLING PROFILE;” and

U.S. Pat. No. 8,285,390 entitled “MONITORING THE COOLING OF SUBCUTANEOUS LIPID-RICH CELLS, SUCH AS THE COOLING OF ADIPOSE TISSUE.”

### TECHNICAL FIELD

The present disclosure relates generally to adhesive liners for cryotherapy applicators. In particular, several embodiments are directed to adhesive liners configured to apply a substance to a subject's skin and associated technology.

### BACKGROUND

Excess body fat, or adipose tissue, may be present in various locations of the body, including, for example, the thighs, buttocks, abdomen, knees, back, face, arms, and other areas. Excess adipose tissue can detract from personal appearance and athletic performance. Moreover, excess adipose tissue is thought to magnify the unattractive appearance of cellulite, which forms when subcutaneous fat lobules protrude or penetrate into the dermis and create dimples where the skin is attached to underlying structural fibrous strands. Cellulite and excessive amounts of adipose tissue are often considered cosmetically unappealing. For example, excess adipose tissue located at a subject's outer thighs can form “saddlebags,” and excess adipose tissue at the sides of the subject's waistline can form “love-handles” or a “muffin top.” Diet and exercise may be insufficient to significantly reduce such excess adipose tissue.

Aesthetic improvement of the human body often involves the selective removal of adipose tissue. Common procedures for this purpose are invasive, such as liposuction or other surgical techniques. Invasive procedures, however, tend to be associated with high cost, long recovery times, and increased risk of complications. In many instances, non-invasive or minimally invasive procedures can allow some or all of these disadvantages to be avoided while providing at least comparable clinical outcomes as those of invasive procedures. For example, non-invasive removal of excess subcutaneous adipose tissue can eliminate both unnecessary recovery time and discomfort associated with invasive procedures, such as liposuction. Conventional non-invasive treatments for removing excess body fat often include application of topical agents, use of weight loss drugs, regular exercise, dieting, or a combination of these treatments. One drawback of these treatments is that they may not be effective or even possible under certain circumstances. For example, when a person is physically injured or ill, regular exercise may not be an option. Similarly, weight loss drugs or topical agents are not an option if, as another example, they cause an allergic or negative reaction. Furthermore, fat loss in selective areas (e.g., inner or outer regions of the thighs) of a person's body often cannot be achieved using general or systemic weight loss methods.

Other methods for non-invasively reducing subcutaneous adipose tissue by cooling are disclosed in U.S. Pat. No. 7,367,341 entitled “METHODS AND DEVICES FOR SELECTIVE DISRUPTION OF FATTY TISSUE BY CONTROLLED COOLING” to Anderson et al. and U.S. Patent Publication No. 2005/0251120 entitled “METHODS AND DEVICES FOR DETECTION AND CONTROL OF SELECTIVE DISRUPTION OF FATTY TISSUE BY CONTROLLED COOLING” to Anderson et al., the entire disclosures of which are incorporated herein by reference. During a procedure, it may be difficult to keep a sufficient amount of coupling gel between a cooling device and a patient's skin. For example, absorbent pads carrying coupling gels are frequently replaced to maintain a desired level of gel between tissue cooling devices and the patient's skin. Additionally, the topical gels can spread along the patient's skin and contaminate the applicator, requiring a significant amount of post-treatment cleanup.

### BRIEF DESCRIPTION OF THE DRAWINGS

In the drawings, identical reference numbers identify similar elements or acts.

FIG. 1 is a partially schematic, isometric view of a treatment system for non-invasively affecting subcutaneous target regions of a subject in accordance with an embodiment of the technology.

FIG. 2 is a cross-sectional view of a connector of the treatment system taken along line 2-2 of FIG. 1.

FIG. 3 shows an adhesive liner positioned under an applicator secured to a subject's thigh in accordance with embodiments of the technology.

FIG. 4 is a cross-sectional view of the adhesive liner and the applicator taken along line 4-4 of FIG. 3.

FIG. 5 is a top view of the adhesive liner in accordance with embodiments of the technology.

FIG. 6 is a bottom view of the adhesive liner of FIG. 5.

FIGS. 7-12 are a series of views of a method for applying a liner and an applicator in accordance with various embodiments of the present technology.

FIG. 13 is a flowchart of a method for treating a subject in accordance with embodiments of the technology.

FIGS. 14-18 are a series of views of a method for treating a subject.

FIGS. 19 and 20 are top and bottom views, respectively, of an adhesive liner in accordance with another embodiment.

FIG. 21 is an isometric view of a treatment isolator in accordance with embodiments of the technology.

FIG. 22 is an isometric view of the treatment isolator of FIG. 21 with a central region removed.

FIG. 23 is a plan view of a treatment isolator in accordance with one embodiment.

FIG. 24 is a side view of the treatment isolator of FIG. 23.

FIG. 25 is a detailed cross-sectional view of a portion of the treatment isolator taken along the line 25-25 of FIG. 24.

FIGS. 26-30 are a series of views of a method for utilizing a treatment isolator to perform a cryotherapy procedure.

FIG. 31 is a schematic block diagram illustrating subcomponents of a control system in accordance with an embodiment of the disclosure.

### DETAILED DESCRIPTION

### Overview

The present disclosure describes liners, treatment isolators, conformable applicators, methods for affecting targeted tissue, and associated technology. Several of the details set forth below are provided to describe the following examples and methods in a manner sufficient to enable a person skilled in the relevant art to practice, make and use them. In some embodiments, a system includes an adhesive liner for applying a topical substance to a treatment region and an applicator for cooling/heating the treatment region. Several of the details and advantages described below, however, may not be necessary to practice certain examples and methods of the technology. Additionally, the technology may include other examples and methods that are within the scope of the technology but are not described in detail.

At least some embodiments of the present technology include treatment systems for affecting tissue in a target region of a human subject's body. The term “treatment system,” as used generally herein, refers to cosmetic or medical treatment systems. The treatment system can reduce or eliminate love handles, saddlebags, muffin tops, or other undesired body features. In various embodiments, the treatment system includes a comfortable adhesive liner and an applicator for conductively heating/cooling targeted tissue. The adhesive liner can contain and apply substances to the target region. A treatment isolator can localize the effects of the therapy to a specific treatment area. Treatment isolators can include one or more thermally-insulating masks, liners (e.g., adhesive liners), or other devices or elements capable of effectively managing heat transfer between an applicator and non-targeted tissue.

The adhesive liner can include one or more features for carrying a cryoprotectant or thermal coupling substance. Such features can include a cavity, an absorbent member (e.g., a cotton pad, gauze, etc.) preloaded with cryoprotectant or thermal coupling substance, a reservoir, or combinations thereof. When the adhesive liner is applied to a patient, it can create a sealed reservoir capable of containing a flowable or non-flowable cryoprotectant or thermal coupling substance, which touches the patient's skin. The integrity of the sealed reservoir can be maintained when an applicator is pressed against the back side of the liner. Suitable exemplary cryoprotectants and thermal coupling substances and processes for implementing cryoprotectants and thermal coupling substances are described in commonly-assigned U.S. Patent Publication No. 2007/0255362. Other suitable substances can include, without limitation, adhesive gels, flowable conductive substances, adhesive gels whose adhesive strength significantly increases as it is cooled from room temperature t a subject treatment temperature, etc. Exemplary adhesive gels are described in U.S. Patent Application No. 62/276,131, which is incorporated by reference in its entirety. Temperature-dependent adhesive gels can be utilized to promote stable contact, including thermal and/or physical contact, between the liner and tissue region because cooling of temperature-dependent adhesives may significantly strengthen adhesion between the subject's skin and the liner. As such, cooled temperature-dependent adhesives can help prevent or inhibit movement of liners, treatment isolators, and/or applicators relative to a treatment site during the treatment session. In some embodiments, temperature-dependent adhesives include freezing point temperature depressants, pH buffers, humectants, surfactants, and/or additives suitable for topically applied substances. The cryoprotectant or thermal coupling substance can be non-flowable and affixed to the sealed reservoir upon manufacture of the liner, or it can be flowable and introduced into the reservoir through a filling port after attachment of the liner to the patient. If it is non-flowable, it can be affixed to a mesh type structure attached to the reservoir or embedded in a hydrogel or affixed in any other conventional manner.

In some embodiments, an adhesive liner for holding a flowable substance in contact with a subject's skin includes a liner body, a first adhesive, and the filling port. The liner body can include a treatment window region for viewing an underlying treatment site. The first adhesive can encircle the treatment window region. The filling port is configured to be in fluid communication with a sealed reservoir between the treatment window region and a subject's skin when the first adhesive couples the liner body to the subject's skin. A flowable substance, which has been delivered through the filling port and contained in the sealed reservoir, can contact the treatment site while a cryotherapy applicator applied to the treatment window region can cool the liner body and an area of the treatment site contacting the flowable substance. In one embodiment, the filling port can have an access end and an outlet end. The access end can be positioned external to the liner body and the outlet end can extend past a section of the first adhesive and into the sealed reservoir when the liner body is coupled to the subject's skin.

The filling port can include a one-way valve that allows the flowable substance to flow into the sealed reservoir when the liner body is coupled to the subject's skin. The filling port can include a tube extending across a section of the first adhesive such that an outlet end of the tube is positioned directly between the treatment window region and the treatment site when the first adhesive couples the liner body to the subject's skin.

The first adhesive can provide a fluid-tight seal with the subject's skin to keep the flowable substance in the sealed reservoir. The adhesive liner can further include a second adhesive positioned on the treatment window region and having a second adhesive strength for adhering to the subject's skin. The first adhesive has a first adhesive strength that is greater than the second adhesive strength. In certain embodiments, the second adhesive strength is substantially less than the strength of the first adhesive strength. In various embodiments, the second adhesive strength equal to or less than about 10%, 20%, 30%, 40%, 50%, 60%, 70%, of 80% of the first adhesive strength.

A flowable substance that is delivered between the liner body and the subject's skin via the filling port can break adhesion between the second adhesive and the skin while the first adhesive provides a fluid-tight seal with the subject's skin for containing the flowable substance within the sealed reservoir. The adhesive liner can further include an applicator adhesive on a backside on the liner body. A cryotherapy applicator can be adhered to a back side of the treatment window region via the applicator adhesive.

In certain embodiments, a liner includes a liner body, a first adhesive, a filling port, and a second adhesive. The filling port is positioned to deliver a flowable substance to a location between the liner body and a subject's skin when the first adhesive couples the liner body to the subject. The second adhesive is positioned on the liner body to adhere a region of the liner body to the subject's skin before the flowable substance is delivered through the filling port. The second adhesive has a second adhesive strength that is less than the first adhesive strength such that the flowable substance delivered between the liner body and the subject's skin breaks adhesion between the second adhesive and the skin while the first adhesive keeps the flowable substance contained between the liner body and the subject's skin. The adhesive liner can include a third adhesive positioned on a backside of the liner body and configured to be adhered to a cryotherapy application.

In some embodiments, a method for treating a subject comprises applying an adhesive liner to form a fluid-tight seal with the subject's skin. A flowable substance is delivered into a sealed space between a front side of the adhesive liner and the subject's skin while the adhesive liner maintains the fluid-tight seal. Skin contacting the flowable substance can be cooled using the cryotherapy applicator, which has been applied to the backside of the adhesive liner.

The adhesive liner can be applied to the subject by pressing a first adhesive of the adhesive liner against the subject's skin to form the fluid-tight seal. A second adhesive of the adhesive liner can be pressed against the subject's skin to adhere a transparent section of the adhesive liner to the subject's skin. The second adhesive is surrounded by the first adhesive and has an adhesion strength substantially less than an adhesion strength of the second adhesive.

Some of the embodiments disclosed herein can be for cosmetically beneficial alterations of a variety of body regions. Some treatment procedures may be for the sole purpose of altering the body region to conform to a cosmetically desirable look, feel, size, shape or other desirable cosmetic characteristic or feature. Accordingly, at least some embodiments of the cosmetic procedures can be performed without providing an appreciable therapeutic effect (e.g., no therapeutic effect), For example, some treatment procedures may not include restoration of health, physical integrity, or the physical well-being of a subject. The cosmetic methods can target subcutaneous regions to change a subject's appearance such as, for example, procedures performed on a subject's saddlebags (i.e., excess adipose tissue at the subject's thighs and/or buttocks) and/or love handles (i.e., excess adipose tissue at the side of a subject's waistline). In other embodiments, however, the cosmetically desirable treatments may have therapeutic outcomes (whether intended or not), such as psychological benefits, alteration of body hormone levels (by the reduction of adipose tissue), etc.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, stages, or characteristics may be combined in any suitable manner in one or more examples of the technology. The headings provided herein are for convenience only and are not intended to limit or interpret the scope or meaning of the technology.

### Cryotherapy

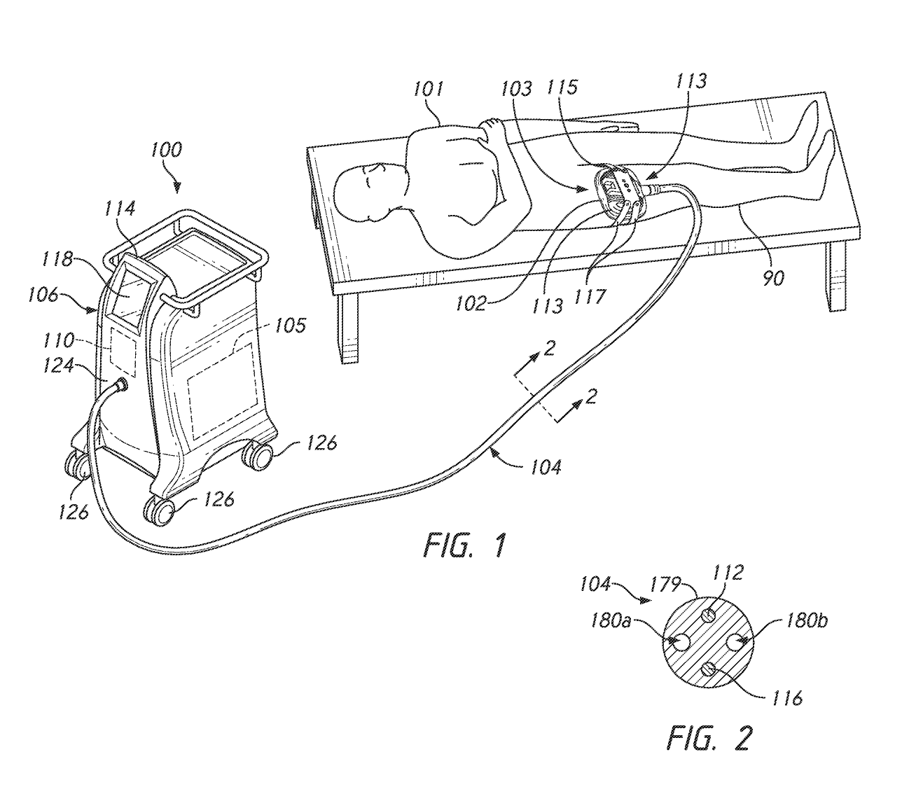

FIG. 1 and the following discussion provide a brief, general description of a treatment system 100 in accordance with some embodiments of the technology. The treatment system 100 can be a temperature-controlled system for cooling/heating tissue of the subject 101. The treatment system 100 can include an adhesive liner 102 (“liner 102”) and an applicator system 103 capable of conforming to contoured treatment sites. The applicator system 103 is positioned along the subject's leg 90 and can include a conformable applicator 113 (“applicator 113”) and a strap assembly 115. The adhesive liner 102 is positioned under the applicator 113 and can be used to deliver substances to the subject's skin while preventing direct physical contact between temperature-controlled surfaces of the applicator 113, and the treatment region and substances on the subject's skin. The strap assembly 115 can wrap around the leg 90 to hold the applicator 113 in thermal contact with a relatively large treatment region.

The illustrated adhesive liner 102 and applicator 113 are positioned to selectively cool subcutaneous, lipid-rich tissue of the subject's right leg 90 to reduce or eliminate a saddlebag, although the adhesive liner 102 and applicator 113 can be applied to other body parts to treat additional treatment sites. The liner 102 can inhibit, limit, or substantially prevent movement of the applicator 113 relative to the treatment site. The substance applied to liner 102 can be liquids, gels, flowable or non-flowable, and can include one or more freezing point temperature depressants, thickening agents, pH buffers, humectants, surfactants, and/or additives suitable for topically application. If the substance is flowable, it can be replenished without removing the liner 102 and/or applicator 113 relative to the patient.

Without being bound by theory, the selective effect of cooling is believed to result in, for example, membrane disruption, cell shrinkage, disabling, damaging, destroying, removing, killing or other methods of lipid-rich cell alteration. Such alteration is believed to stem from one or more mechanisms acting alone or in combination. It is thought that such mechanism(s) trigger an apoptotic cascade, which is believed to be the dominant form of lipid-rich cell death by non-invasive cooling. In any of these embodiments, the effect of tissue cooling is to selectively reduce lipid-rich cells by a desired mechanism of action, such as apoptosis, lipolysis, or the like. In some procedures, the applicator system 103 can cool the skin of the patient to a temperature in a range of from about −30° C. to about 20° C., from about −20° C. to about 20° C., from about −15° C. to about 10° C., or from about −15° C. to about −5° C. In some embodiments, the temperatures of the skin or applicator can be from about −20° C. to about 10° C., from about −18° C. to about 5° C., from about −15° C. to about 5° C., or from about −15° C. to about 0° C. In further embodiments, the cooling temperatures can be equal to or less than −5° C., −10° C., −15° C., or in yet another embodiment, from about −15° C. to about −25° C. Other cooling temperatures can be used.

Apoptosis, also referred to as “programmed cell death”, is a genetically-induced death mechanism by which cells self-destruct without incurring damage to surrounding tissues. An ordered series of biochemical events induce cells to morphologically change. These changes include cellular blebbing, loss of cell membrane asymmetry and attachment, cell shrinkage, chromatin condensation and chromosomal DNA fragmentation. Injury via an external stimulus, such as cold exposure, is one mechanism that can induce cellular apoptosis in cells. Nagle, W. A., Soloff, B. L., Moss, A. J. Jr., Henle, K. J. “Cultured Chinese Hamster Cells Undergo Apoptosis After Exposure to Cold but Nonfreezing Temperatures” Cryobiology 27, 439-451 (1990).

One aspect of apoptosis, in contrast to cellular necrosis (a traumatic form of cell death causing local inflammation), is that apoptotic cells express and display phagocytic markers on the surface of the cell membrane, thus marking the cells for phagocytosis by macrophages. As a result, phagocytes can engulf and remove the dying cells (e.g., the lipid-rich cells) without eliciting an immune response. Temperatures that elicit these apoptotic events in lipid-rich cells may contribute to long-lasting and/or permanent reduction and reshaping of subcutaneous adipose tissue.

One mechanism of apoptotic lipid-rich cell death by cooling is believed to involve localized crystallization of lipids within the adipocytes at temperatures that do not induce crystallization in non-lipid-rich cells. The crystallized lipids may selectively injure these cells, inducing apoptosis (and may also induce necrotic death if the crystallized lipids damage or rupture the bi-lipid membrane of the adipocyte). Another mechanism of injury involves the lipid phase transition of those lipids within the cell's bi-lipid membrane, which results in membrane disruption or dysfunction, thereby inducing apoptosis. This mechanism is well-documented for many cell types and may be active when adipocytes, or lipid-rich cells, are cooled. Mazur, P., “Cryobiology: the Freezing of Biological Systems” Science, 68: 939-949 (1970); Quinn, P. J., “A Lipid Phase Separation Model of Low Temperature Damage to Biological Membranes” Cryobiology, 22: 128-147 (1985); Rubinsky, B., “Principles of Low Temperature Preservation” Heart Failure Reviews, 8, 277-284 (2003). Other possible mechanisms of adipocyte damage, described in U.S. Pat. No. 8,192,474, relate to ischemic/reperfusion injury that may occur under certain conditions when such cells are cooled as described herein. For instance, during treatment by cooling, the targeted adipose tissue may experience a restriction in blood supply and thus be starved of oxygen due to isolation as a result of applied pressure, cooling which may affect vasoconstriction in the cooled tissue, or the like. In addition to the ischemic damage caused by oxygen starvation and the buildup of metabolic waste products in the tissue during the period of restricted blood flow, restoration of blood flow after cooling treatment may additionally produce reperfusion injury to the adipocytes due to inflammation and oxidative damage that is known to occur when oxygenated blood is restored to tissue that has undergone a period of ischemic. This type of injury may be accelerated by exposing the adipocytes to an energy source (via, e.g., thermal, electrical, chemical, mechanical, acoustic, or other means) or otherwise increasing the blood flow rate in connection with or after cooling treatment as described herein. Increasing vasoconstriction in such adipose tissue by, e.g., various mechanical means (e.g., application of pressure or massage), chemical means or certain cooling conditions, as well as the local introduction of oxygen radical-forming compounds to stimulate inflammation and/or leukocyte activity in adipose tissue may also contribute to accelerating injury to such cells. Other yet-to-be understood mechanisms of injury may exist.

In addition to the apoptotic mechanisms involved in lipid-rich cell death, local cold exposure is also believed to induce lipolysis (i.e., fat metabolism) of lipid-rich cells and has been shown to enhance existing lipolysis which serves to further increase the reduction in subcutaneous lipid-rich cells. Vallerand, Zamecnik. J., Jones, P. J. H., Jacobs, I. “Cold Stress Increases Lipolysis, FFA Ra and TG/FFA Cycling in Humans” Aviation, Space and Environmental Medicine 70, 42-50 (1999).

One expected advantage of the foregoing techniques is that the subcutaneous lipid-rich cells in the target region can be reduced generally without collateral damage to non-lipid-rich cells in the same region. In general, lipid-rich cells can be affected at low temperatures that do not affect non-lipid-rich cells. As a result, lipid-rich cells, such as those associated with cellulite, saddlebags, love handles, muffin tops, etc., can be affected while other cells in the same region are generally not damaged even though the non-lipid-rich cells at the surface (e.g., cells in the dermis and/or epidermis) may be subjected to even lower temperatures than those to which the lipid-rich cells are exposed.

In a typical procedure, the applicator system 103 can remove heat from the underlying tissue through the upper layers of the skin and create a thermal gradient with the coldest temperatures near the cooling surface of the applicator system 103 (i.e., the temperature of the upper layer(s) of the skin can be lower than that of the targeted underlying cells). It may be challenging to reduce the temperature of the deep cells (e.g., lipid-rich cells) low enough to be destructive to these target cells (e.g., induce apoptosis, cell death, etc.) while also maintaining the temperature of the upper and surface skin cells high enough so as to be protective (e.g., non-destructive). The temperature difference between these two thresholds can be small (e.g., about 5° C. to about 10° C., less than 10° C., less than 15° C., etc.). Protection of the overlying cells (e.g., typically water-rich dermal and epidermal skin cells) from freeze damage during dermatological and related aesthetic procedures that require sustained exposure to cold temperatures may include improving the freeze tolerance and/or freeze avoidance of these skin cells. Cryoprotectants can be applied using the liner 102 to inhibit, minimize, or prevent such freeze damage.

Additionally, mechanical vibratory energy can be imparted to the patient's tissue to effect therapy. In some embodiments, an applicator can have one or more vibrators, such as unbalanced masses, rotating masses, or the like, to vibrate the tissue before, during, and/or after a cooling period. The liner 102 can remain securely coupled to the patient to prevent or limit leaking of the topically applied substance even when significant pressure is applied and/or during massaging. Additionally or alternatively, other types of energy can be used to effect therapy, Example energies include, without limitation: ultrasound, electromagnetic energy, radiofrequency, thermal energy, or the like. The characteristics of the components of the system can be selected to achieve desired energy delivery to the patient.

### Treatment Systems

FIG. 1 shows the treatment system 100 including the applicator system 103, a connector 104, and a control module 106. The connector 104 can provide energy (e.g., electrical energy) and fluid (e.g., coolant) from the control module 106 to the applicator system 103. The strap assembly 115 can include flexible straps 117 that can be positioned to comfortably hold the applicator 113, which can be a non-vacuum based cooling device capable of cooling subcutaneous tissue without pinching, thus allowing treatment of generally non-pinchable regions, such as large volume fat bulges (e.g., saddlebags), abdominal regions, flank regions, etc.

FIG. 2 is a cross-sectional view of the connector 104 in accordance with at least some embodiments of the technology. The connector 104 can include a main body 179, a supply fluid line or lumen 180a (“supply fluid line 180a”), and a return fluid line or lumen 180b (“return fluid line 180b”). The main body 179 may be configured (via one or more adjustable joints) to “set” in place for the treatment of the subject 101 and can include a multi-lumen hose, a covering, a sheath, or other components for protecting electrical/fluidic lines. The supply and return fluid lines 180a, 180b can be conduits comprising, in whole or in part, polyethylene, polyvinyl chloride, polyurethane, and/or other materials that can accommodate circulating coolant, such as water, glycol, synthetic heat transfer fluid, oil, a refrigerant, and/or any other suitable heat conducting fluid. In one embodiment, each fluid line 180a, 180b can be a flexible hose surrounded by the main body 179. The connector 104 can also include one or more electrical lines 112 (one illustrated schematically in FIG. 2) for providing power to the applicator 113 and a control line 116 (one illustrated schematically in FIG. 2) for providing communication between the control module 106 (FIG. 1) and the applicator 113 (FIG. 1). In various embodiments, the connector 104 can include a bundle of fluid conduits, a bundle of power lines, wired connections, and other bundled and/or unbundled components. The configuration of the connector 104 can be selected to provide ergonomic comfort, minimize unwanted motion (and thus potential inefficient removal of heat from the subject 101), and/or to provide an aesthetic appearance to the treatment system 100.

Referring again to FIG. 1, the control module 106 can include a fluid chamber 105 (illustrated in phantom line), a power supply 110 (illustrated in phantom line), and a controller 114 carried by a housing 124 with wheels 126. The control module 106 can include a refrigeration unit, a cooling tower, a thermoelectric chiller, heaters, or any other device capable of controlling the temperature of coolant in the fluid chamber 105. The coolant can be continuously or intermittently delivered to the applicator 113 via the supply fluid line 180a (FIG. 2) and can circulate through the applicator 113 to absorb heat. The coolant, which has absorbed heat, can flow from the applicator 113 back to the control module 106 via the return fluid line 180b (FIG. 2). For warming periods, the control module 106 can heat the coolant such that warm coolant is circulated through the applicator 113. Alternatively, a municipal water supply (e.g., tap water) can be used in place of or in conjunction with the control module 106.

An operator can control operation of the treatment system 100 using an input/output device 118 of the controller 114. The power supply 110 can provide a direct current voltage for powering electrical elements of the applicator 113 via the line 112 (FIG. 2). The controller 114 can monitor process parameters based on output from sensors (e.g., sensors placed proximate to the applicator 113, sensors of the applicator 113, etc.) communicated via the control line 116 (FIG. 2). In some embodiments, the controller 114 can exchange data with the applicator 113 via a wireless or optical communication link. The controller 114 can monitor and adjust treatment based on one or more treatment profiles and/or patient-specific treatment plans, such as those described, for example, in commonly assigned U.S. Pat. No. 8,275,442. Each custom treatment profile can include one or more segments, and each segment can include a specified duration, a target profile, or the like. For example, a treatment profile for reducing love-handles or saddlebags can include specified treatment sites, specified durations for each treatment site, and/or target temperature profiles for each treatment site. In some cryotherapy sessions, saddlebags located on opposite sides of a subject's body are treated in the same session using the same applicator or multiple applicators. A different liner can be used at each treatment site to facilitate post-treatment cleanup. Additionally, a treatment profile can include specific temperature profiles for each cooling unit of the applicator. Exemplary individually-controlled heat-exchanging cooling units are described herein and additional applicators and cooling units are described in commonly assigned U.S. Patent Publication Nos. 2008/0077211 and 2011/0238051.

### Adhesive Liners

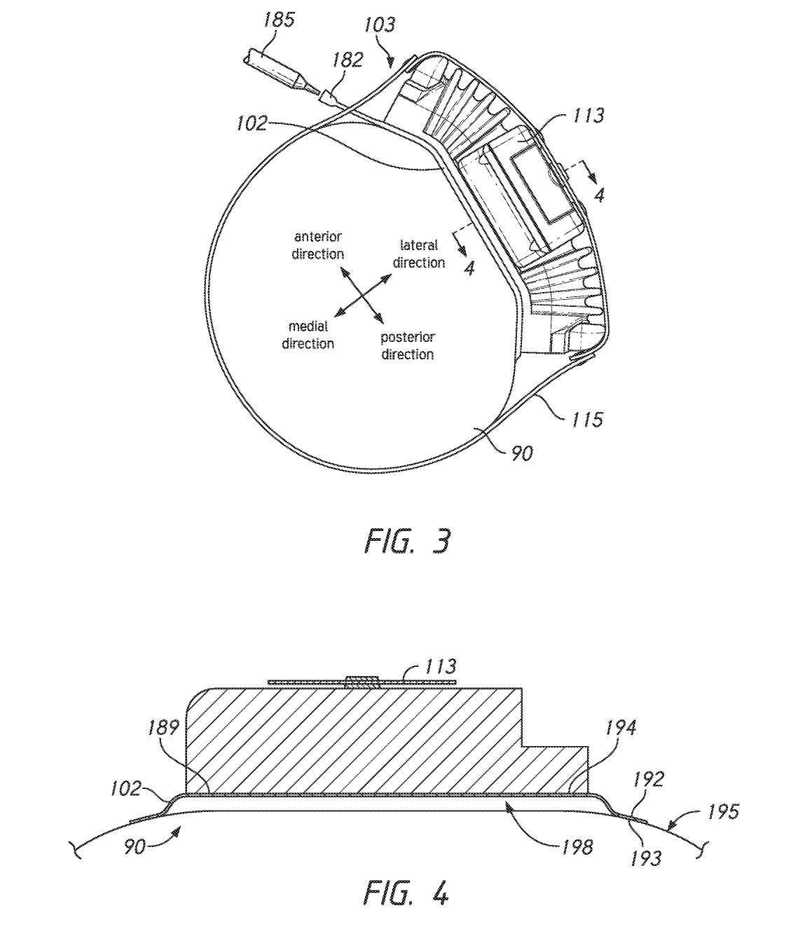

FIG. 3 shows the liner 102 positioned under the applicator 113 in accordance with embodiments of the technology. FIG. 4 is a cross-sectional schematic view of the liner 102 and applicator 113 taken along line 4-4 of FIG. 3. Referring now to FIG. 3, the liner 102 has a filling port 182 in fluid communication with a sealed reservoir 198 (FIG. 4) located between the liner 102 and the subject's skin when the liner 102 is attached to the subject. A dispenser 185 can be mated with the filling port 182 and then operated to deliver a flowable substance into the reservoir 198 (FIG. 4). The liner 102 contains the flowable substance while preventing direct physical contact between the applicator 113 and the patient. This can simplify cleanup after completion of the treatment session. If the treatment period is relatively long (e.g., longer than 20 minutes, 30 minutes, 40 minutes, or 50 minutes), the subject may want to periodically move his/her leg 90. The liner 102 and strap assembly 115 can cooperate to maintain thermal contact between the target tissue and applicator 113 independent of the patient repositioning his/her leg 90. Additionally, the applicator 113 can be adhered to the liner 102 to inhibit, limit, or substantially prevent movement of the applicator 113.

Referring now to FIG. 4, the liner 102 can include a compliant liner body 189 with an outer periphery 192 and a treatment window region 194. The outer periphery 192 can be adhered to the subject's skin 195 to define the reservoir 198. A fluid-tight seal 193 can be maintained to prevent leaking of the flowable substance, including when the applicator 113 is pressed against the liner 102.

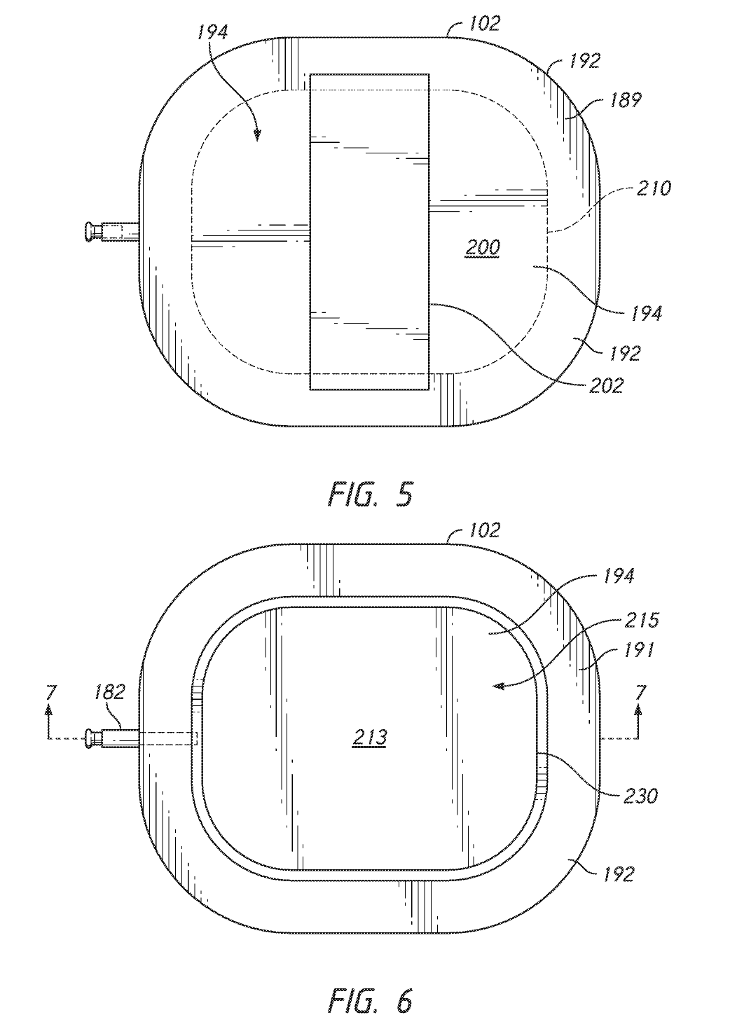

FIG. 5 is a top view of the liner 102 in accordance with embodiments of the present technology. FIG. 6 is a bottom view of the liner 102 of FIG. 5. Referring now to FIG. 5, the liner body 189 can include an applicator-contact side or backside surface 200 (“backside surface 200”) with an applicator adhesive 202 that comprises, in whole or in part, one or more pressure sensitive adhesives or suitable adhesives for contacting temperature-controlled surfaces of the cryotherapy applicator. After the applicator is adhered to the liner 102, the sides of the applicator laterally adjacent to the adhesive 202 can freely move along the backside surface 200 such that the applicator can assume a wide range of configurations.

The liner body 189 can include applicator-positioning indicium or indicia 210 (illustrated in dashed line) applied via printing, embossing, or another suitable technique. In some embodiments, the applicator-positioning indicium or indicia 210 (“applicator-positioning indicia 210”) can be located along the boundary of the treatment window region 194 (“treatment window 194”) and can have a shape complementary to the shape of the cryotherapy applicator. An operator can utilize the applicator-positioning indicia 120 to help position the applicator relative to the target site. For example, an applicator can be positioned inside, and centered with or otherwise positioned with respect to, the indicia 210. The shape and size of the area defined by the applicator-positioning indicia 210 can be selected based on the configuration of the applicator and desired positioning accuracy.

The entire liner body 189 can be made of optically-transparent silicon, rubber, or polymer, such as a flexible thermoplastic polyester ether (TPE) which is tear-resistant under normal operating temperatures. In one embodiment, the periphery 192 can be made, in whole or in part, of an opaque material, and the treatment window 194 can comprise an optically-transparent or semi-transparent material. In certain embodiments, the treatment window 194 can be located inside the applicator-positioning indicia 210 and can be made of a transparent material and/or a semi-transparent material.

FIG. 6 shows a patient side or surface 213 of the liner 102. The surface 213 has a reservoir-defining region 215 generally corresponding to the treatment window 194. An adhesive 191 can encircle the treatment window 194 and, in some embodiments, can be an adhesive strip extending along most of or an entire periphery of the liner body 189. The adhesive 191 can be a high-strength acrylic adhesive suitable for forming fluid-tight seals with skin. An adhesive 230 can be positioned along the treatment window 184 and can have adhesive characteristics suitable for facilitating adhesion of the liner 102. In some embodiments, the adhesive 230 can be a double-sided adhesive suitable for skin contact. Example adhesives include low-tack silicon adhesives, acrylic adhesives, or combinations thereof. In various embodiments, the adhesive strength of the adhesive 230 is equal to or less than about 10%, 20%, 30%, 40%, 50%, 60%, 70%, of 80% of adhesive strength of the adhesive 191. The location and characteristics of the adhesives can be selected based on the procedures to be performed.

FIGS. 7-12 show a series of views of a method for applying the liner 102 in accordance with embodiments of the present technology. FIG. 7 is a cross-sectional view of the liner 102 with release liners 400, 402, 404 covering respective adhesives 191, 230, 202. The filling port 182 has an inlet or access end 420 (“access end 420”), an outlet end 422, and a main body 440. The access end 420 can protrude outwardly from the liner body 189 and can be configured to engage a needle, a tapered end, or another feature of a dispenser, a line, etc. In some embodiments, the filling port 182 can include, without limitation, a female Luer fitting, a male Luer fitting, or another connection. The main body 440 can be a tube that is adhered, bonded, welded, or otherwise coupled to the main body 189, and layers 450, 452 can be adhered, bonded, welded, or otherwise coupled together to the filling port 182. The filling port 182 can include at least one valve 460 to control the flow of a substance. The valve 460 can be, for example, a check valve, a duckbill valve, or another one-way valve that allows substances to flow in one direction along a flow path 462. In other embodiments, the valve 460 is a two-way valve that selectively allows substances to flow into and out of the reservoir. The features, configuration, and functionality of the filling port 182 can be selected based on the characteristics of the flowable substance.

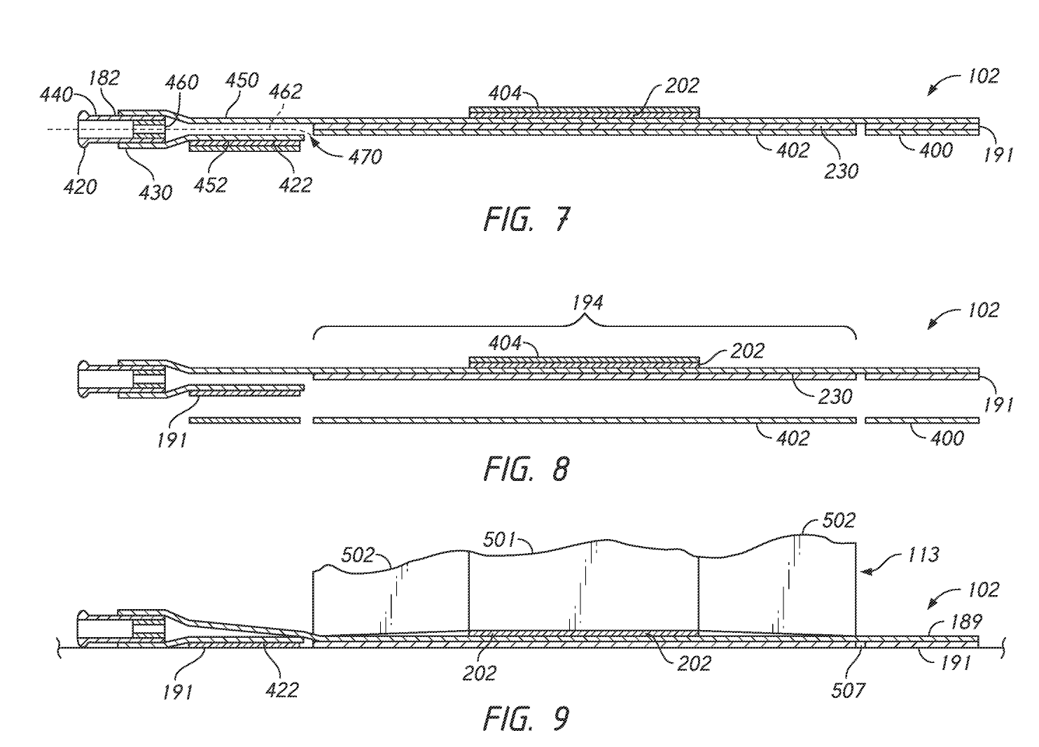

FIG. 8 is a cross-sectional view of the liner 102 after removing the front side liners 400, 402. When the liner 102 is positioned generally above the treatment area, the treatment site can be viewed via the treatment window 194. Air between the liner 102 and patient can be removed by manually pressing along the backside of the liner 102 until achieving desired adhesion. Before and/or after adhering the liner 102 to the subject, the backside liner 404 can be separated from the adhesive 202. An applicator can then be applied to the adhesive 202.

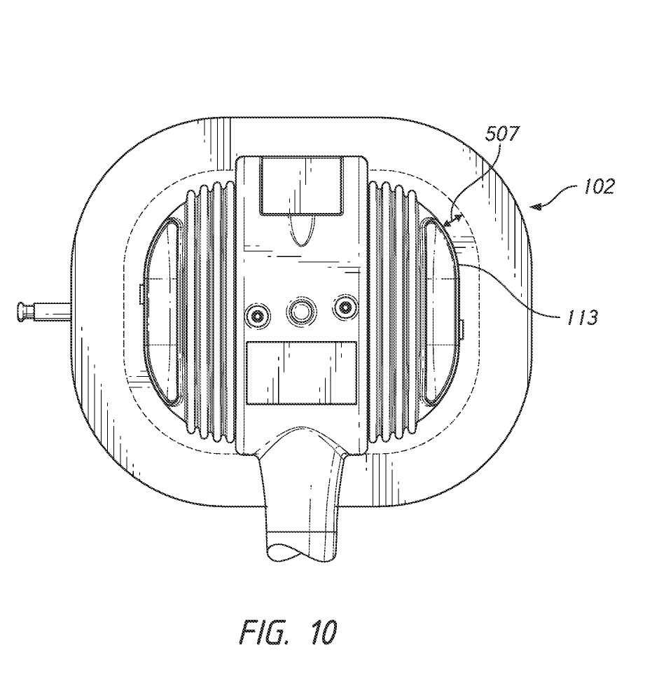

FIGS. 9 and 10 are cross-sectional and top views, respectively, of the applicator 113 applied to the adhesive liner 102 in accordance with various embodiments. Referring to FIG. 9, a central section 501 of the applicator 113 can be adhered to the main body 189. The outlet end 422 can extend past the adhesive 191 a distance selected to limit or prevent disruption of the adhesive 191 when flowable substances are delivered through filling port 182. As shown in FIG. 10, the applicator 113 can be spaced apart from an inner periphery (indicated in dashed line) of the adhesive 191, such that when the flowable substance is delivered under the liner 102, air can be pushed outwardly past the periphery of temperature-controlled surfaces of the applicator 113 and can become trapped in a gap 507, which generally between the applicator 113 and the adhesive 191 (FIG. 9). When the applicator 113 is generally centered with the liner 102, the gap 507 can have a generally uniform width along most of the perimeter of the applicator 113. The small gap 507 of FIG. 10 can contain a small volume of trapped air whereas the large gap 507 of FIG. 10 can contain a relatively large volume of trapped air. The gap 507 can have a width of about 1 cm, 2 cm, 3 cm, 4 cm, 5 cm, 6 cm, 7 cm, or another with can be selected based on the expected volume of trapped air.

FIG. 11 shows the dispenser 185 coupled to the filling port 182 and the reservoir 198 partially filled with a substance 521. The adhesive 230 can have an strength (e.g., adhesive strength, peel strength, shear strength, seal strength, etc.) that is less than the strength of the adhesive 191. This allows the flowable substance 521 to controllably break, for example, adhesion between the adhesive 230 and the skin 195 while the adhesive 191 maintains a seal 226 (e.g. a liquid-tight seal, a gas-tight seal, etc.). Air trapped in the reservoir 198 can be pushed outwardly past the periphery of the applicator 113.

The substance 521 can help maintain thermal contact between the temperature-controlled surface(s) of the applicator 113. The substance 521 can include one or more cryoprotectants as described in commonly-assigned U.S. Patent Publication No. 2007/0255362. Other substances can be used and can include, without limitation, adhesive gels, including exemplary adhesive gels described in U.S. Patent Application Nos. 62/276,131; 62/334,337; 62/334,213; Ser. Nos. 11/741,271; 14/108,807, which are incorporated by reference in their entireties. For example, temperature-dependent adhesive gels can be utilized to promote stable contact, including thermal and/or physical contact, between the liner and tissue region. Cooling of the temperature-dependent adhesives may significantly strengthen adhesion between the subject's skin and the liner and prevent any movement therebetween during a patient treatment. In some embodiments, temperature-dependent adhesives include freezing point temperature depressants, thickening agents, pH buffers, humectants, surfactants, combinations thereof, or other additives.

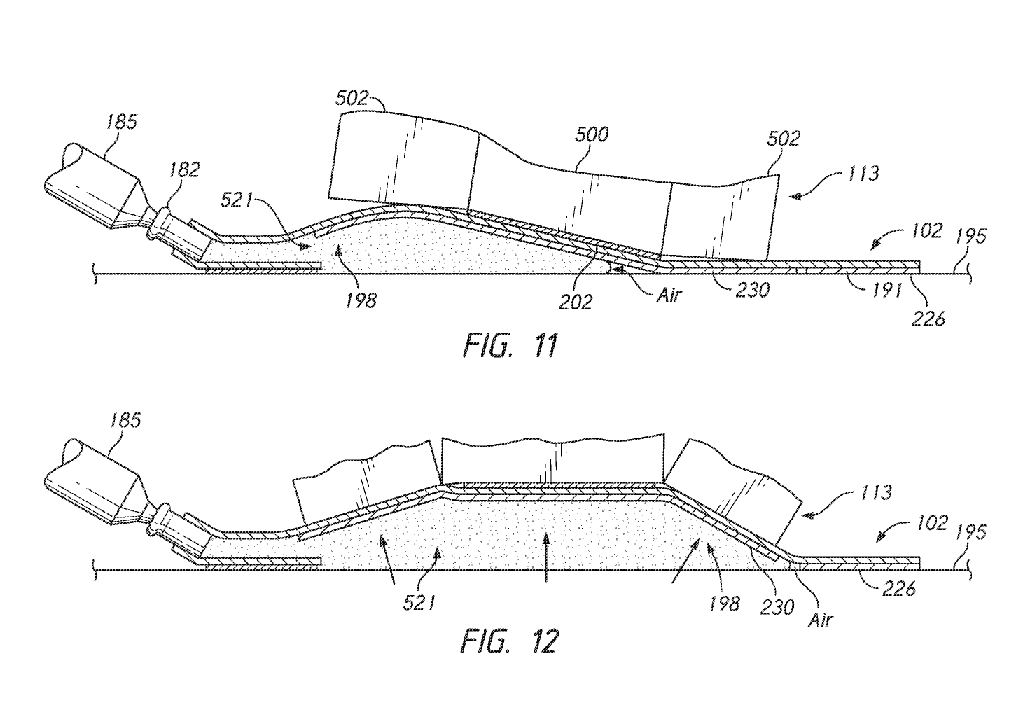

FIG. 12 shows the sealed cavity or reservoir 198 filled with the substance 521. Thermal energy (indicated by arrows) can be transferred across the substance 521 and the liner 102. Air, which can act as an insulator, can be kept outside of the thermal path to enhance heat transfer and increase the effectiveness of therapy. This produces efficient heat transfer for rapid cooling/heating.

### Methods of Treatment

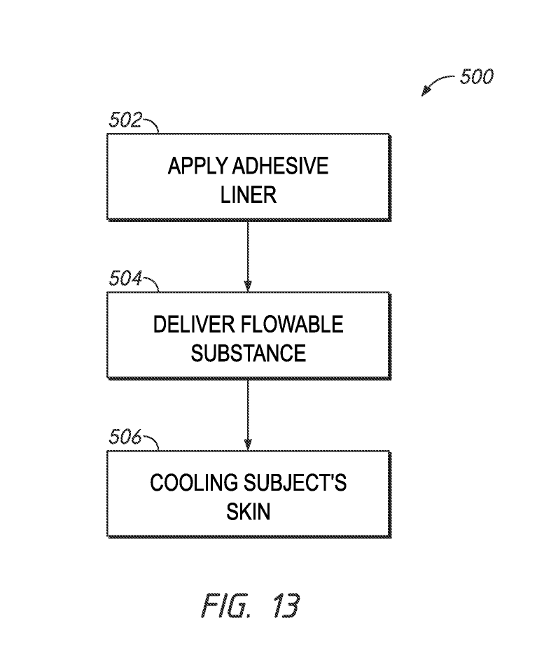

FIG. 13 is a flowchart of a method 500 for treating a subject in accordance with embodiments of the present technology. At block 502, release liners are removed from the adhesive liner 102, and the liner 102 is applied to a treatment site, as discussed in connection with FIGS. 7-9. At block 504, a substance can be delivered into the sealed space between a front side of the adhesive liner 102 and the subject's skin, as discussed in connection with FIGS. 11 and 12. At block 506, a cryotherapy applicator is applied to a backside of the adhesive liner 102 and a strap assembly can be used to hold the cryotherapy applicator. The adhesive liner 102 can keep the substance in physical contact with the subjects skin to, for example, protect tissue (e.g., shallow non-targeted tissue), enhance the effects of cooling, or combinations thereof.

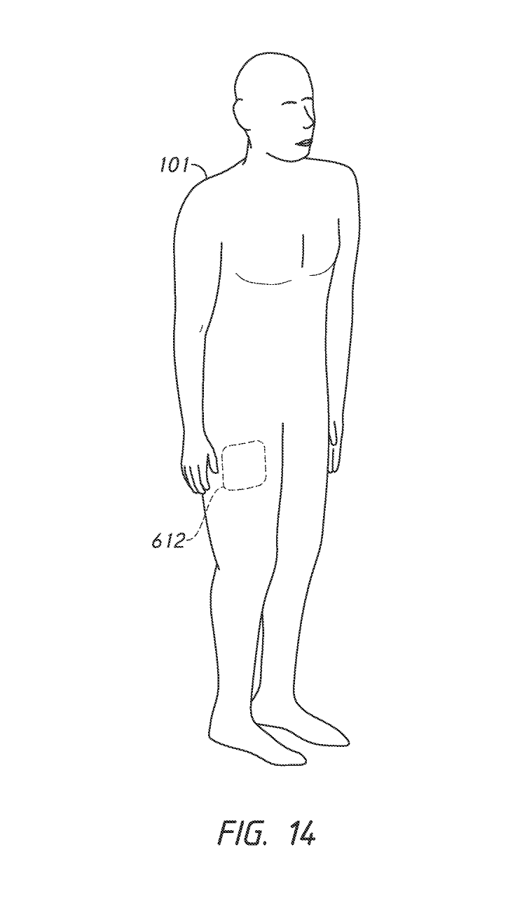

FIGS. 14-18 depict a series of views of the method for treating a subject. FIG. 14 shows a standing patient 101 with a marked treatment region 612. The region 612 can be identified using stickers, tape, markers, or the like while the patient stands, because patients often want an optimal aesthetic appearance when they are upright (e.g., standing, walking, running, etc.). The patient can be evaluated in other positions if desired.

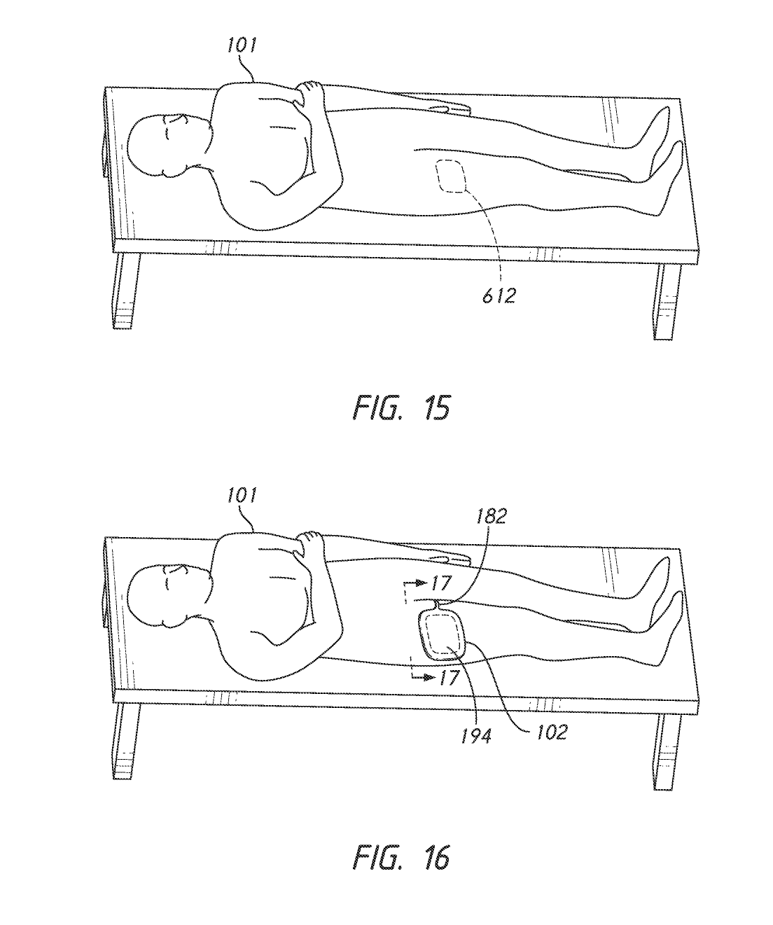

FIG. 15 shows the subject 101 ready to be treated. Pretreatment skin wipes, cleaning solutions, and other pretreatment substances can be used to prepare the treatment region 612. The liner 102 can be applied to the subject's skin using, for example, techniques discussed in connection with FIGS. 7-9.

FIG. 16 shows the adhesive liner 102 applied to the subject 101 such that the treatment window 194 is generally centered with the treatment region 612. A substance can then be delivered under the liner 102.

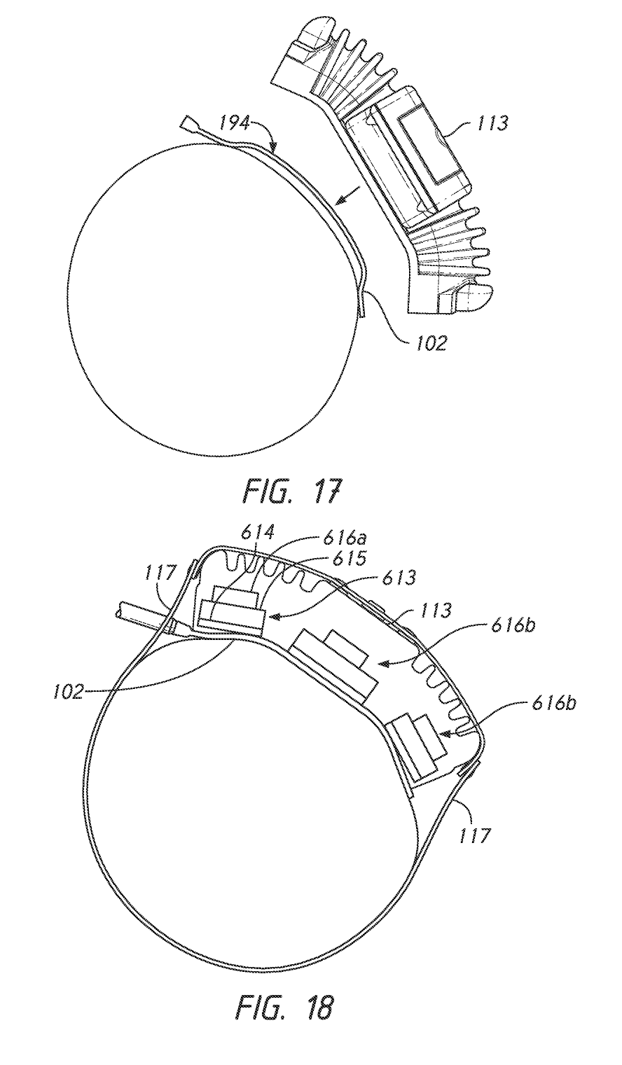

FIG. 17 shows the applicator 113 ready to be applied to the liner 102. The periphery of the applicator 113 can be aligned with the treatment window 194, positioning indicia, and/or another feature of the adhesive liner 102. Once aligned, the applicator 113 can be pressed against the adhesive liner 102.

FIG. 18 shows the liner 102 compressed between the applicator 113 and the subject's leg. The applicator 113 can include one or more heat-exchanging units 613 (one identified) for cooling/heating tissue. Each heat-exchanging unit 613 can include one or more heating/cooling elements, thermoelectric devices (e.g. Peltries devices), and/or other components for heating/cooling tissue. The applicator 113 can have flat or curved plates 614 made of metal or other conductive material (e.g., a rigid conductive material, a flexible conductive material, etc.), and may be covered by a film, a sheet, a sleeve, or other component suitable for defining an interface surface. Cooling plates 614 can be flat, curved, concave, convex, wavy, or the like. In some embodiments, the cooling plates 614 can have radius of curvature in one or more directions (e.g., a radius of curvature in one direction, a first radius of curvature in a first direction and a second radius of curvature in a second direction, etc.). In one embodiment, a rigid or flexible heat-exchanging element can have a radii of curvature in a direction generally parallel to the length or width of its exposed surface. Additionally, each heat-exchanging element can have the same configuration. In other embodiments, the heat-exchanging elements can have different configurations. The plates 614 can be heated/cooled by thermoelectric devices 615. Fluid-cooled devices 616 can exchange heat with the backside of the thermoelectric devices 615 to keep the thermoelectric devices at or below target temperature. The illustrated applicator 113 includes three heat-exchanging units 613. The number, positions, and configurations of the heat-exchanging units 613 can be selected based on the desired temperature profile to be achieved. The shapes, dimensions, and properties, such as mechanical properties, thermal properties, or the like, of the heat-exchanging elements and other components of the applicators can be selected to achieve the desired interaction with the subject.

With continued reference to FIG. 18, the straps 117 can be individually tensioned to pull the applicator 113 towards the thigh. The applied pressure can help tissue to conform to the applicator 113 while the applicator 113 assumes the general shape (e.g., curvature) of the body surface. The straps 117 can be used to adjust the pressure distribution applied by the applicator 113 and can be independently positioned along the subject's specific anatomy. The conformability of the compliant liner 102 and applicator 113 results in a large area of thermal contact and a comfortable fit. Because the liner 102 prevents direct contact between the applicator 113 and the subject's skin, the likelihood of cross-contamination between patients is reduced, as well as minimizing post-treatment cleaning requirements for the applicator 113. Additional liners or protective sleeves may be used to provide sanitary barriers and interfaces.

The liner 102 can prevent or limit movement of the applicator 113 relative to subject's skin without utilizing, for example, a vacuum. As such, the applicator 113 can be used at treatment sites not suitable for drawing tissue into vacuum cups. Additionally, a region can be treated without treating all, or most of, the circumference of the subject's body part to comfortably treat local sites, such as non-pinchable fat bulges.

To treat saddlebags, the cooling unit 616a can be generally positioned on an anterior side of the saddlebag and another cooling unit 616c can be positioned on a posterior side of a saddlebag. The central cooling unit 616b can be positioned generally along the middle of the saddlebag. The straps 117 can be tensioned to compress tissue and eliminate gaps between the applicator 113 and the subject. The applicator 113 can cycle through, for example, segments of a prescribed saddlebag treatment plan, which can include one segment for reducing the saddlebag on one side of the subject and another segment for reducing the other saddlebag. In other treatment plans, multiple applicators 113 can simultaneously treat saddlebags on opposite sides of the subject.

During treatment, the system can determine whether a temperature or heat flux is sufficiently close to the target temperature or heat flux based, at least in part, on measurements from one or more temperature sensors. It will be appreciated that while a region of the body has been cooled or heated to the target temperature, in actuality the temperature of that body region may be close but not equal to the target temperature because of the body's natural heating and cooling variations. Thus, although the system may attempt to heat or cool the tissue to the target temperature or to provide a target heat flux, one or more sensors may measure a sufficiently close temperature or heat flux. If the target temperature has not been reached, power can be increased or decreased to change the heat flux to maintain the target temperature or “set-point” selectively to affect targeted tissue. When the prescribed segment duration expires, the controller may apply the temperature and duration indicated in the next treatment profile segment. In some embodiments, temperature can be controlled using a variable other than, or in addition to, power. For example, the controller can provide cooling to the target region based on a predetermined or real-time determined treatment protocol.

The applicator 113 can include additional features for providing a vacuum, energy (e.g., electrical energy, radiofrequency, ultrasound energy, thermal energy, etc.), and so forth. In vacuum embodiments, the treatment systems can include a pressurization device that assists in forming a contact between the applicator and the liner 102 applying a vacuum. In one embodiment, mechanical vibratory energy can be imparted to the patient's tissue by repeatedly applying and releasing a vacuum to the subject's tissue, for instance, to create a massage action during treatment. Further details regarding vacuum type devices and operation may be found in U.S. Patent Application Publication No. 2008/0287839. In one embodiment, the applicator 113 can include one or more vibrators, rotating unbalanced masses, etc.

The liner 102 may also include or incorporate one or more sensors (e.g., pressure sensors, temperature sensors, contact sensors, etc.) and various storage, computing, and communications devices, such as a radio frequency identification (RFID) component, allowing for example, use to be monitored and/or metered. Exemplary features usable with liners and applicators are described in commonly assigned U.S. Patent Publication No. 2008/0077201. Liners may be designed to treat certain portions of the patient's body, such as saddlebags, love-handles, chin, cheeks, arms, pectoral areas, thighs, calves, buttocks, abdomen, back, and so forth and can be sufficiently conformable to achieve a desired amount of thermal contact. Exemplary components and features that can be incorporated into the systems disclosed herein are described in, e.g., commonly assigned U.S. Pat. No. 7,854,754 and U.S. Patent Publication Nos. 2008/0077201, 2008/0077211, 2008/0287839, 2011/0238050, and 2011/0238051.

Liners with Air-Trapping Features

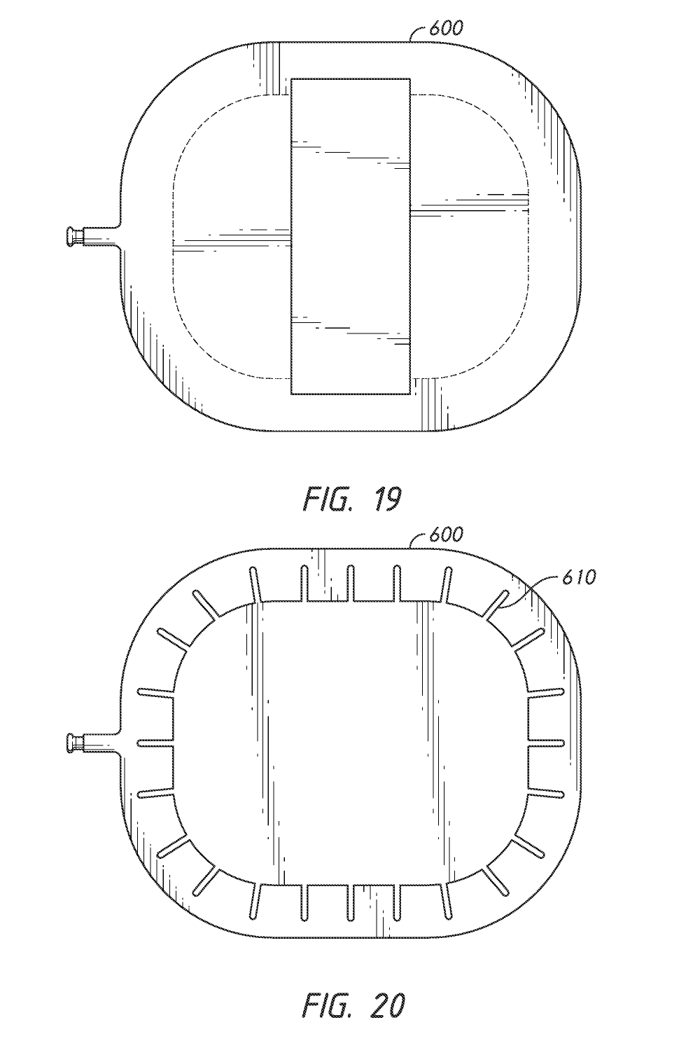

FIGS. 19 and 20 are top and bottom views, respectively, of an adhesive liner 600 in accordance with another embodiment. The description of the liner 102 applies equally to the adhesive liner 600, except as detailed below. The liner 600 can include features in the form of air-trapping features 610 (one identified in FIG. 20) configured to limit or reduce the amount of air located directly between the applicator and the subject's treatment region. The air-trapping features 610 can be channels, grooves, or other features for receiving air while a flowable substance is delivered to the liner 600. To vent air, the air-trapping features 610 can include valves that selectively allow trapped air to escape. After removing the air, the valves can be closed. The configuration (e.g., straight, serpentine, arcuate, etc.), number, sizes, and positions of the air-trapping features can be selected based on areas in which air tends to be trapped. Although the features 610 are discussed with respect to trapping air, the features 610 can collect the flowable substance and provides other functionality.

### Treatment Isolators

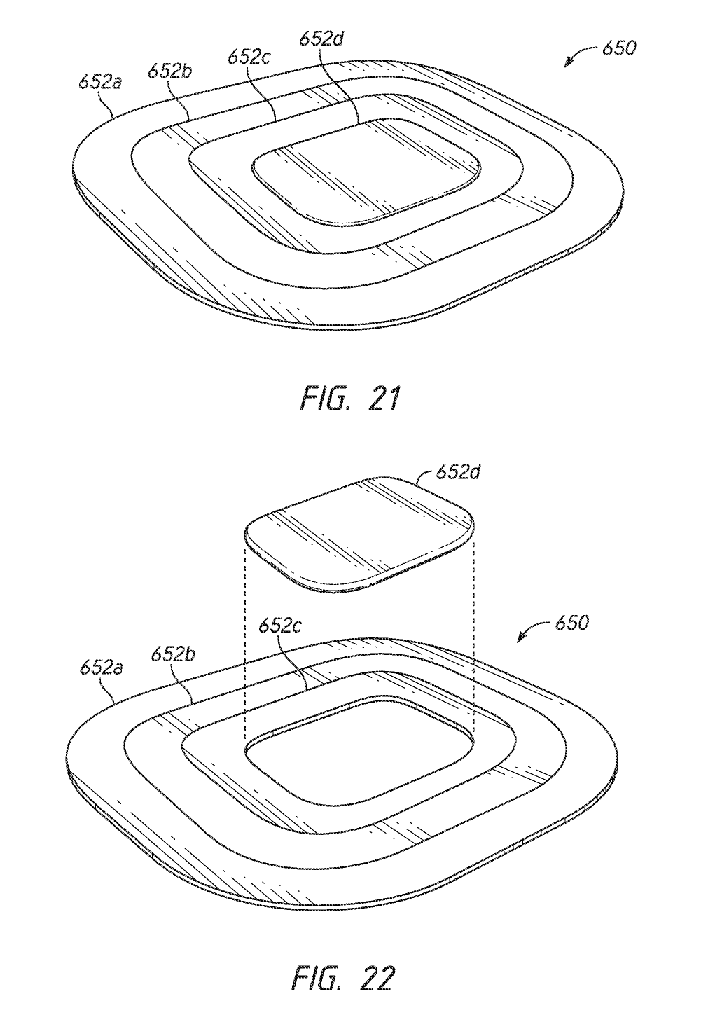

FIG. 21 is an isometric view of a treatment isolation device in the form of a treatment or thermal isolator 650 (“thermal isolator 650”). Generally, the thermal isolator 650 has an adhesive for coupling to a liner, an applicator, and/or directly to a patient's body and can include one or more removable regions 652a, 652b, 652c, 652d (collectively, “regions 652”). FIG. 22 shows the thermal isolator 650 with the innermost region 652d removed. The other regions 652a-c can be positioned between an applicator and the subject to inhibit heat-transfer to limit or prevent non-targeted tissue from being affected by the therapy. Other regions 652 can be removed to provide an opening or window that matches the treatment area such that regions 652 applied to the patient's body can inhibit heat transfer with underlying non-target tissue while targeted tissue is cooled via the window.

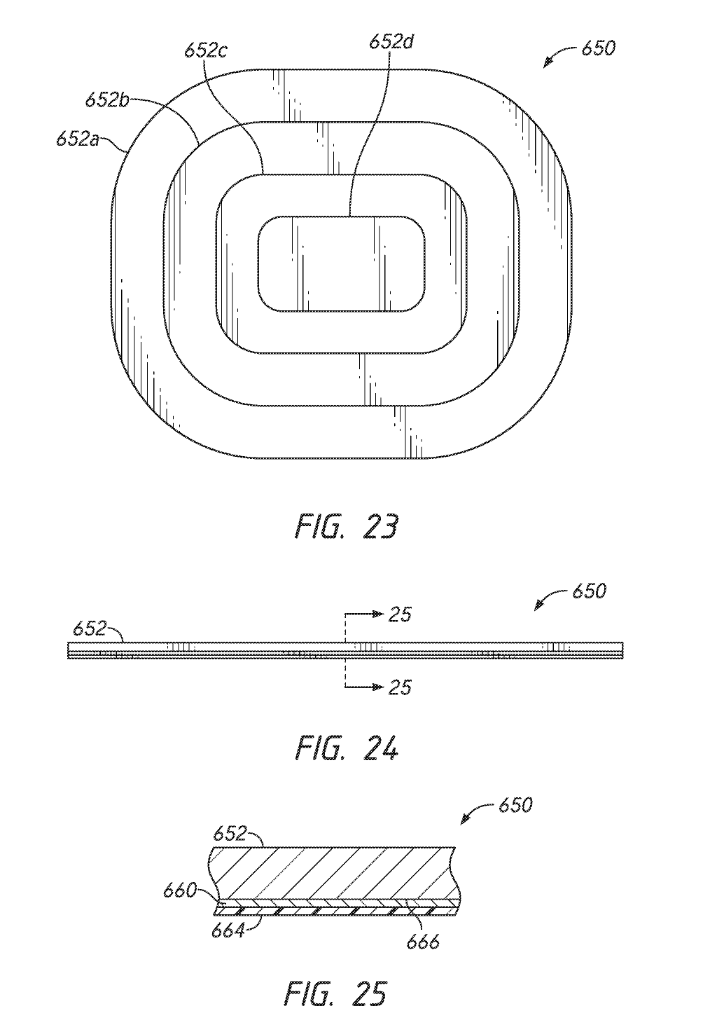

FIG. 23 is a top view of the thermal isolator 650 in accordance with one embodiment. The regions 652 can have any shape, including, but not limited to, polygonal shapes (e.g., rectangular shapes, square shapes, or the like), circular shapes, elliptical shapes, irregular shapes, or other suitable shapes. In some embodiments, the regions 652a, 652b, 652c can be nested rounded rectangular rings, and the region 652d can be a rounded rectangle. This allows a very large applicator to be used to cool/heat a wide range of relatively small treatment sites with different dimensions. Break lines, scoring, or weakened regions can allow the regions to be torn apart from one another. In embodiments suitable for use with cutting instruments, markings can be printed on the thermal isolator and a cutting instrument can be used to remove regions of the thermal isolator.

FIG. 24 is a side view of the thermal isolator 650, and FIG. 25 is a detailed cross-sectional side view of the thermal isolator 650. Referring now to FIG. 25, the thermal isolator 650 can include an adhesive 660 and a release liner 664. The adhesive 660 can include one or more adhesive gels, binding agents, and/or pressure sensitive adhesives and can be disposed on a bottom surface 666 of the regions 652, and the release liner 664 can cover the adhesive 660.

The thermal isolator 650 can comprise, in whole or in part, closed-cell foam, open-cell foam, rubber, silicon, combinations thereof, or another thermally insulating material. The thickness of the thermal isolator 650 can be equal to or greater than, for example, 0.5 mm, 1 mm, 2 mm, 5 mm, 10 mm, 15 mm, or another desired thickness and can have a uniform or varying thickness. The thermal properties, dimensions, and mechanical properties of the treatment isolator can be selected to maintain non-targeted tissue at or above a selected temperature, such as about −5° C., −1° C., 0° C., 5° C., 10° C., 20° C., 25° C., 30° C., or the like.

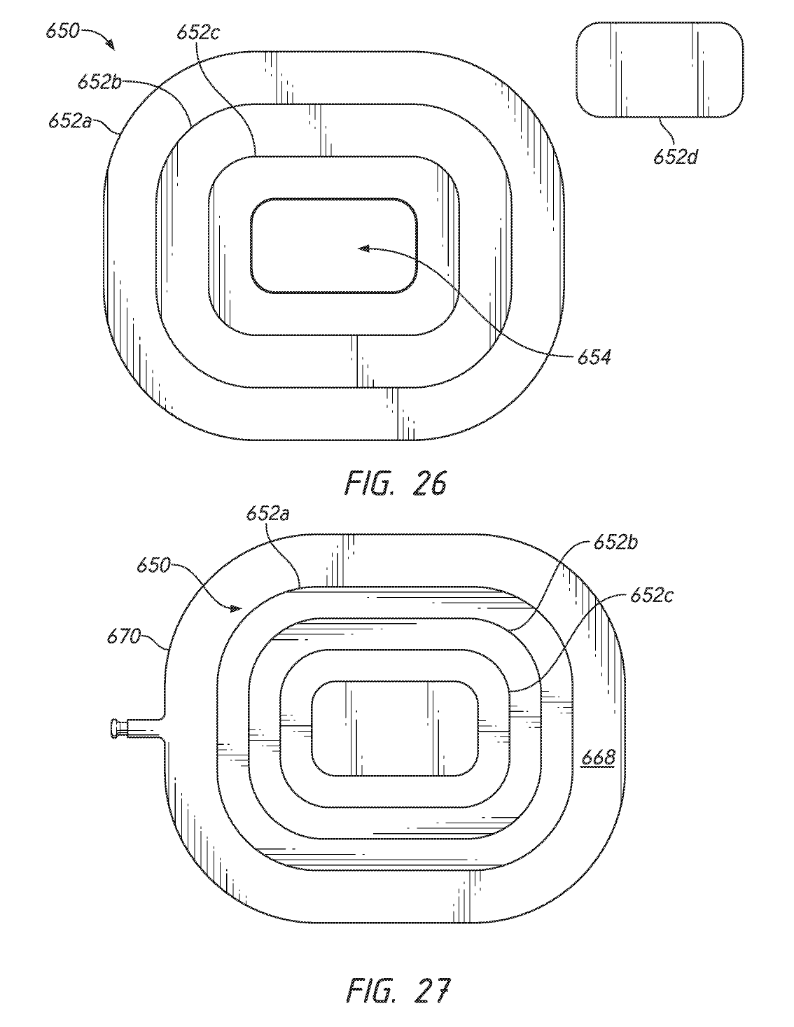

FIGS. 26-30 are a series of views of a method for using the thermal isolator 650. FIG. 26 shows the innermost region 652d after it has been removed. FIG. 27 shows the thermal isolator 650 after release liners have been removed from the remaining regions 652a, 652b, 652c and the thermal isolator 650 has been adhered to a backside 668 of an adhesive liner 670, which can include positioning features (e.g., markings) used to align the thermal isolator 650.

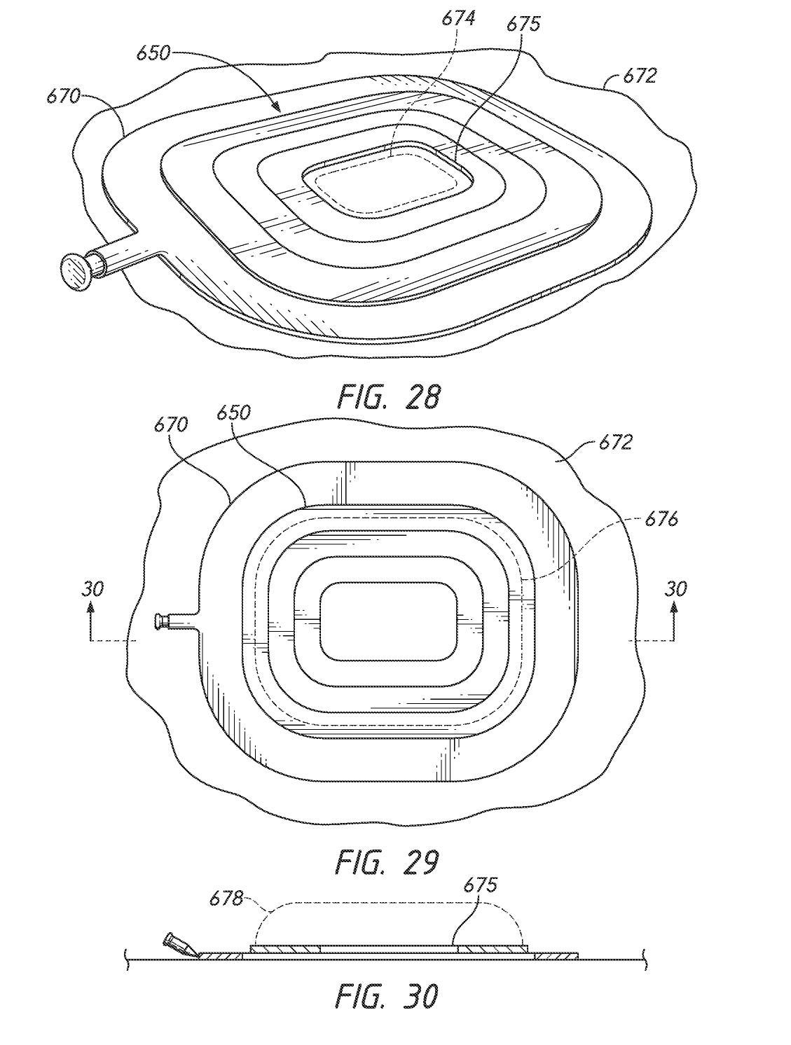

FIG. 28 is an isometric view of the liner 670 and the thermal isolator 650 positioned on a subject 672. A target treatment area 674 (indicated in dashed line) can be positioned in the window 675, and if the area 674 is a bulge caused by excess adipose tissue, it may extend through the window 675. In a single-segment treatment session, the entire region with excess adipose tissue can be located within the window 675, and in a multi-segment treatment session, different areas of the subject's body can be sequentially positioned in the window 675. The configuration and size of the window can be selected based on the number of segments in a treatment session, number of treatment sessions, and/or additional treatment parameters.

FIG. 29 is a top view of the liner 670 and thermal isolator 650 applied to the subject 672. FIG. 30 is a cross-section view of the liner 670 and thermal isolator 650 taken along line 30-30 of FIG. 29. Referring now to FIG. 29, an applicator can be placed upon the thermal isolator 650 at the applicator position 676 (indicated in dashed line) such that thermal isolator 650 and/or liner 670 extend outwardly past the periphery of the applicator.

FIG. 30 shows an applicator 678 (shown in dashed line) positioned on the thermal isolator 650. As a topically substance is delivered underneath or into the liner 670, a section of a liner or thermal isolator 650 can be moved upwardly through the window 675 and into contact with temperature-controlled surface(s) the applicator 678. As such, the window 675 can help establish a thermal path between the applicator 678 and the subject's targeted skin.

Treatment isolators can also be attached directly to applicators, which are then placed on the liner or directly on the subject. In some procedures, sections of thermal isolators can be selectively removed and the thermal isolator can be adhered directly to temperature controlled surfaces of the applicator. In yet other procedures, treatment isolators can be adhered directly to the subject's skin, and a liner can then be placed on the treatment isolator. Tissue exposed by the thermal isolator can be contacted by a substance contained by the liner while the subject's skin underlying the thermal isolator does not physically contact the substance and is thermally insulated. Treatment isolators can be used in other procedures to achieve desired temperature profiles across a region of the subject's body. Optionally, the liner may not have an adhesive, and in some other procedures, no liner is used. For example, a treatment isolator can be directly applied to the subject's skin, and an applicator can be directly applied to a backside of the treatment isolator.

Treatment isolators can also have sections with different thermal conductivities. A highly conductive region can be used to conductively cool target tissue while insulated regions can protect non-targeted tissue. This allows for varying temperature profiles along a patient's skin when using a uniform temperature cooling surface. In one embodiment, a treatment isolator can be a sheet with discrete regions having thermal conductivities selected to achieve a desired temperature profile. Another technique for protecting non-targeted tissue can include applying a spreadable protective thermally-insulating substance. A physician can visually inspect the subject's body and determine the boundary between targeted and non-targeted tissue. The non-targeted tissue can then be covered with a thermally-insulating spreadable material that serves as a thermal isolator. In some embodiments, the physician configures the thermal isolator by visually inspecting the subject to identify the treatment region and, based at least in part on the visual inspection, removes a section of the thermal isolator that corresponds to the treatment region.

### Computing Environments

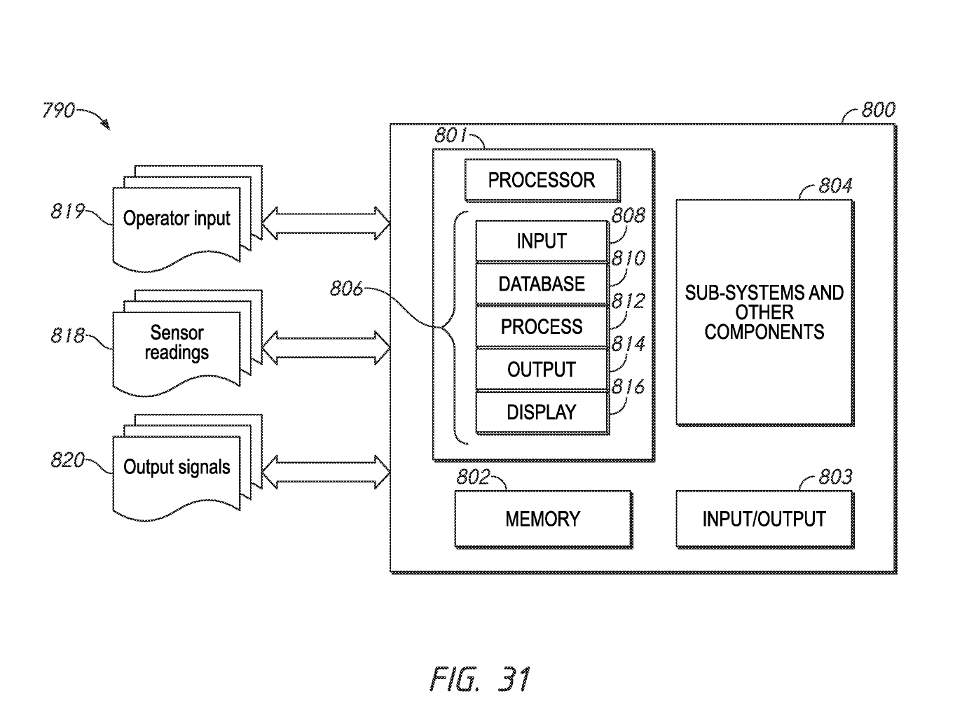

FIG. 31 is a schematic block diagram illustrating subcomponents of a controller in accordance with an embodiment of the disclosure. The controller 790 can be the controller 114 of FIG. 1 or can be incorporated into the other components, such as the applicators disclosed herein. The controller 790 can include a computing device 800 containing a processor 801, a memory 802, input/output devices 803, and/or subsystems and other components 804. The computing device 800 can perform any of a wide variety of computing processing, storage, sensing, imaging, and/or other functions. Components of the computing device 800 may be housed in a single unit or distributed over multiple, interconnected units (e.g., though a communications network). The components of the computing device 800 can accordingly include local and/or remote memory storage devices and any of a wide variety of computer-readable media.

As illustrated in FIG. 31, the processor 801 can include a plurality of functional modules 806, such as software modules, for execution by the processor 801. The various implementations of source code (i.e., in a conventional programming language) can be stored on a computer-readable storage medium or can be embodied on a transmission medium in a carrier wave. The modules 806 of the processor can include an input module 808, a database module 810, a process module 812, an output module 814, and, optionally, a display module 816.

In operation, the input module 808 accepts an operator input 819 via the one or more input devices, and communicates the accepted information or selections to other components for further processing. The database module 810 organizes records, including patient records, treatment data sets, treatment profiles and operating records and other operator activities, and facilitates storing and retrieving of these records to and from a data storage device (e.g., internal memory 802, an external database, etc.). The patient records can include information about liners, applicator operation, sensor readings, etc. Any type of database organization can be utilized, including a flat file system, hierarchical database, relational database, distributed database, etc.

In the illustrated example, the process module 812 can generate control variables based on sensor readings 818 from sensors and/or other data sources, and the output module 814 can communicate operator input to external computing devices and control variables to the controller. The display module 816 can be configured to convert and transmit processing parameters, sensor readings 818 (signals from sensors), output signals 820, input data, treatment profiles and prescribed operational parameters through one or more connected display devices, such as a display screen, printer, speaker system, etc. The sensor readings 818 can be signals from sensors in a liner (e.g., liner 102, liner 670), sensors in thermal isolators (e.g., isolator 650), sensors in an applicator, etc. Exemplary sensors include, without limitation, temperature sensors, heat flux sensors, pressure sensors, and/or contact sensors.

In various embodiments, the processor 801 can be a standard central processing unit or a secure processor. Secure processors can be special-purpose processors (e.g., reduced instruction set processor) that can withstand sophisticated attacks that attempt to extract data or programming logic. The secure processors may not have debugging pins that enable an external debugger to monitor the secure processor's execution or registers. In other embodiments, the system may employ a secure field-programmable gate array, a smartcard, or other secure devices.

The memory 802 can be standard memory, secure memory, or a combination of both memory types. By employing a secure processor and/or secure memory, the system can ensure that data and instructions are both highly secure and sensitive operations such as decryption are shielded from observation. In various embodiments, the memory 802 can be flash memory, secure serial EEPROM, secure field programmable gate array, or secure application-specific integrated circuit.

The input/output device 803 (e.g., input/output device 118 of FIG. 1) can include, without limitation, a keyboard, a mouse, a stylus, a push button, a switch, a potentiometer, a scanner, an audio component such as a microphone, or any other device suitable for accepting user input and can also include one or more video monitors, a medium reader, an audio device such as a speaker, any combination thereof, and any other device or devices suitable for providing user feedback. For example, if the applicator 113 moves an undesirable amount during a treatment session, the input/output device 803 can alert the subject 101 and/or operator via an audible alarm. The input/output device 118 can be a touch screen that functions as both an input device and an output device. The control panel can include visual indicator devices or controls (e.g., indicator lights; numerical displays, etc.) and/or audio indicator devices or controls. The control panel may be a component separate from the input device 118 and/or output device 120, may be integrated with one or more of the devices, may be partially integrated with one or more of the devices, may be in another location, and so on. In alternative embodiments, the controller 114 can be contained in, attached to, or integrated with the applicator 113. In yet other embodiments, the various components can be fixedly installed at a treatment site. Further details with respect to components and/or operation of applicators, control modules (e.g., treatment units), and other components may be found in commonly-assigned U.S. Patent Publication No. 2008/0287839.

The controller 790 can include any processor, Programmable Logic Controller, Distributed Control System, secure processor, or the like. A secure processor can be implemented as an integrated circuit with access-controlled physical interfaces; tamper resistant containment; means of detecting and responding to physical tampering; secure storage; and shielded execution of computer-executable instructions. Some secure processors also provide cryptographic accelerator circuitry. Suitable computing environments and other computing devices and user interfaces are described in commonly assigned U.S. Pat. No. 8,275,442, entitled “TREATMENT PLANNING SYSTEMS AND METHODS FOR BODY CONTOURING APPLICATIONS,” which is incorporated herein in its entirety by reference.

### CONCLUSION

Although noninvasive applicators are illustrated and discussed herein, minimally invasive applicators may also be employed. As an example, a cryoprobe, electrode, and/or other invasive component may be incorporated into the applicators disclosed herein and can be inserted directly into the targeted tissue (e.g., subcutaneous adipose tissue) to cool, freeze, or otherwise thermally process the targeted tissue. Moreover, cooling tissue and related devices and systems may be disclosed herein primarily or entirely in the context of cryolipolysis and cryolysis, but other contexts in addition to those disclosed herein are within the scope of the present invention. It should be understood, in general, that other methods, devices, and systems in addition to those disclosed herein are within the scope of the present invention. For example, methods, devices, and systems in accordance with embodiments of the present invention can have different and/or additional configurations, components, and procedures than those disclosed herein. Moreover, a person of ordinary skill in the art will understand that methods, devices, and systems in accordance with embodiments of the present invention can be without one or more of the configurations, components, and/or procedures disclosed herein without deviating from the present invention.

The liners, treatment isolators, applicators, retainer systems, strap assemblies, and/or other components of the treatment systems disclosed herein can be included in a kit. In some embodiments, a kit includes single-use disposable components, such as a disposable liners, thermal isolators, retainer system, a disposable cryoprotection element, and/or a disposable holder assembly. The kit can also include instruction documentation containing information regarding how to (a) apply the composition to a target region and/or a heat-exchanging surface of the treatment applicator and (b) reduce a temperature of the target region such that lipid rich cells in the region are affected while preserving non-lipid rich cells proximate to a heat-exchanging surface.

Various embodiments of the technology are described above. It will be appreciated that details set forth above are provided to describe the embodiments in a manner sufficient to enable a person skilled in the relevant art to make and use the disclosed embodiments. Several of the details and advantages, however, may not be necessary to practice some embodiments. Additionally, some well-known structures or functions may not be shown or described in detail, so as to avoid unnecessarily obscuring the relevant description of the various embodiments. Although some embodiments may be within the scope of the technology, they may not be described in detail with respect to the Figures. Furthermore, features, structures, or characteristics of various embodiments may be combined in any suitable manner. Moreover, one skilled in the art will recognize that there are a number of other technologies that could be used to perform functions similar to those described above. While processes or blocks are presented in a given order, alternative embodiments may perform routines having stages, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel, or may be performed at different times. The headings provided herein are for convenience only and do not interpret the scope or meaning of the described technology.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.).

Any patents, applications and other references, including any that may be listed in accompanying filing papers, are incorporated herein by reference. Aspects of the described technology can be modified, if necessary, to employ the systems, functions, and concepts of the various references described above to provide yet further embodiments. These and other changes can be made in light of the above Detailed Description. While the above description details certain embodiments and describes the best mode contemplated, no matter how detailed, various changes can be made. Implementation details may vary considerably, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated.

What is claimed is:

1. An adhesive liner for holding a thermal coupling substance in contact with a subject's skin for use with a cryotherapy cooling applicator for cooling tissue transcutaneously, the adhesive liner comprising:
a liner body including a treatment window region for viewing an underlying treatment site of the subject;
a first adhesive encircling the treatment window region for securing the liner body to the skin along an entire periphery of the window region; and
a thermal coupling substance located within a sealed reservoir between a skin surface at the treatment site and a bottom surface of the liner body facing the skin surface when the first adhesive couples the liner body to the subject's skin, the substance conducting heat from the skin surface while the liner is cooled by the cryotherapy cooling applicator.

2. The adhesive liner of claim 1, wherein the thermal coupling substance includes either a freezing point depressant substance, a freeze protection substance that minimizes damage to skin if it is frozen, an ice nucleating substance that promotes freezing, and/or a temperature dependent adhesive gel whose adhesive strength significantly increases as it is cooled from room temperature to a subject treatment temperature.

3. The adhesive liner of claim 2, wherein the thermal coupling substance includes the temperature dependent adhesive gel which secures the liner to the skin at treatment temperatures.

4. The adhesive liner of claim 1, wherein the first adhesive provides a fluid-tight seal with the subject's skin to keep the substance in the sealed reservoir between the bottom surface of the liner body and the skin surface while the cryotherapy applicator is held against the treatment region.

5. The adhesive liner of claim 1, further comprising an adhesive strip comprising the first adhesive, and wherein the adhesive strip extends along an entire periphery of the liner body.

6. The adhesive liner of claim 1, further comprising a second adhesive located at an applicator side of the treatment region for securing the liner body to the cryotherapy applicator.

7. The adhesive liner of claim 1, further comprising a removable thermal isolator disposed on a top surface of the liner body having a low coefficient of thermal conductivity to inhibit any direct cooling of the subject's skin beneath the thermal isolator by the cryotherapy applicator, the thermal isolator surrounding the treatment region.

8. The adhesive liner of claim 1, further comprising a filling port configured to be in fluid communication with the sealed reservoir so that a substance, which is flowable, can be delivered through the filling port and contained in the sealed reservoir.

9. The adhesive liner of claim 8 wherein the filling port has an access end and an outlet end, and wherein the access end is positioned external to the liner body and the outlet end extends past a section of the first adhesive and into the sealed reservoir.

10. The adhesive liner of claim 8 wherein the filling port includes a one-way valve that allows the flowable substance to flow into the sealed reservoir.

11. The adhesive liner of claim 8 wherein the filling port includes a tube extending across a section of the first adhesive such that an outlet end of the tube is positioned directly between the treatment window region and the treatment site when the first adhesive couples the liner body to the subject's skin.

12. The adhesive liner of claim 8, further comprising a second adhesive positioned on the treatment window region and having a second adhesive strength for adhering to the subject's skin prior to introducing the flowable substance via the filing port, whereby when the flowable substance is introduced a bond between the second adhesive and the subject's skin is broken to form the sealed reservoir into which the flowable substance flows, and wherein the first adhesive has a first adhesive strength that is greater than the second adhesive strength, the first adhesive maintaining a bond between the applicator body and the subject's skin after the flowable substance is introduced, and further comprising a plurality of air trapping features around a perimeter of the liner for trapping air removed from the reservoir as the flowable substance is introduced.

13. The adhesive liner of claim 1 wherein the first adhesive is located on a front side of the liner body, the adhesive liner further comprising an applicator adhesive on a backside on the liner body and configured to adhere the cryotherapy applicator to a backside of the treatment window region when the cryotherapy is applied to the backside of the liner body.

14. The adhesive liner of claim 1 wherein the liner body includes
a patient surface having a reservoir-defining region, and
an applicator surface opposite the patient surface and having an applicator-contact region, and
at least one applicator-placement indicium for positioning the cryotherapy applicator over the reservoir-defining region.

15. The adhesive liner of claim 8, further comprising:
a first release liner covering the first adhesive;
a second release liner covering a second adhesive located at subject side of the treatment window region; and
a third release liner covering a third adhesive located at an applicator side of the treatment window region.

16. A method for treating a subject, comprising:
applying an adhesive liner to a subject's skin, the adhesive liner having a first adhesive around its perimeter to form a fluid-tight seal between the subject's skin and the liner perimeter so as to form a sealed space between a bottom side of the adhesive liner and the subject's skin, the sealed space including a thermal coupling delivering a flowable substance into a sealed space between a front side of the adhesive liner and the subject's skin while the adhesive liner maintain substance; and
cooling the subject's skin, which contacts the thermal coupling substance in the sealed space, using a cryotherapy applicator applied to a backside of the adhesive liner.

17. The method of claim 16 wherein the thermal coupling substance is flowable, and wherein applying the adhesive liner to the subject's skin includes:
pressing a first adhesive of the adhesive liner against the subject's skin to form the fluid-tight seal; and
pressing a second adhesive of the adhesive liner against the subject's skin to adhere a transparent section of the adhesive liner to the subject's skin, wherein the second adhesive is surrounded by the first adhesive and has an adhesion strength substantially less than an adhesion strength of the second adhesive, and further comprising breaking adhesion between the transparent section and the subject's skin provided by the second adhesive by delivering the flowable substance into the sealed space.

18. The method of claim 16, further comprising:
applying the cryotherapy applicator to an applicator adhesive located on the backside of the adhesive liner; and
cooling the backside of the adhesive liner using the cryotherapy applicator to cool the subject's skin while the thermal coupling substance contacts the subject's skin.

19. The method of claim 16, further comprising:
positioning a thermal isolator on the adhesive liner;
applying the cryotherapy applicator to the thermal isolator to establish thermal communication between a target region and at least one temperature controlled surface of the cryotherapy applicator; and
cooling the target region using the cryotherapy applicator while the thermal isolator inhibits cooling of non-targeted tissue adjacent to the target region.

20. The method of claim 16, further comprising:
configuring a thermal isolator for surrounding a treatment region of the subject;
applying the thermal isolator to the adhesive liner; and
cooling the treatment region using the cryotherapy applicator while the thermal isolator inhibits heating/cooling of non-targeted tissue provided by the cryotherapy applicator.
